(19) United States
(12) Patent Application Publication
(10) Pub. No.: US 2024/0216434 A1
(43) Pub. Date: Jul. 4, 2024

(54) NUTRITIONAL COMPOSITIONS FOR HUMAN CONSUMPTION AND METHODS FOR MAKING SAME

(71) Applicant: MJN U.S. Holdings LLC, Evansville, IN (US)

(72) Inventors: John Daniel Alvey, Evansville, IN (US); Zihua AO, Evansville, IN (US); Minhthy Le NGUYEN, Woodland, CA (US)

(73) Assignee: MJN U.S. Holdings LLC, Evansville, IN (US)

(21) Appl. No.: 18/558,050

(22) PCT Filed: Apr. 29, 2022
(86) PCT No.: PCT/EP2022/061636

§ 371 Date: Oct. 30, 2023

Related U.S. Application Data
(60) Provisional application No. 63/182,209, filed on Apr. 30, 2021.

(51) Int. Cl. A61K 35/20; A23J 3/08; A23L 33/00; A61K 31/661

(52) U.S. Cl. CPC A61K 35/20; A23J 3/08; A23L 33/40; A61K 31/661

(57) ABSTRACT

A synthetic nutritional composition for human consumption is described comprising a whey protein concentrate from bovine milk, wherein the whey protein concentrate comprises: a milk fat content of between 6.5 to 10.0 wt %, wherein the milk fat contains components of milk fat globule membranes that provide at least 7 mg/g of sphingomyelin to the whey protein concentrate; and wherein the majority of the sphingomyelin in the synthetic nutritional composition is provided by the components of the milk fat globule membranes of the whey protein concentrate. Methods of making same are also described.

### FIELD OF THE INVENTION

The present invention generally relates to synthetic nutritional compositions for administration to humans which contain certain phospholipids, sphingolipids and, preferably a predetermined whey:casein ratio and methods for making same and particularly, but not exclusively, nutritional compositions for administration to paediatric subjects including a child or an infant.

### BACKGROUND

Synthetic nutritional compositions for human consumption based on bovine dairy products are, typically, made using skimmed milk powder as the predominant dairy component. Skimmed milk powder contains small quantities of bioactive components such as phospholipids, sphingolipids and gangliosides. Some of these bioactive components have been shown to have a profound and lasting impact on human brain function, particularly on the brain function of infants and children whilst they are developing. Sphingomyelin is a type of sphingolipid found in human cell membranes, especially in the membranous myelin sheath that surrounds some nerve cell axons.

Sphingomyelin has been shown in studies to play an important supporting role in myelination which accounts for a large portion of brain growth during the first two years of life. Whilst skimmed milk powder contains these beneficial bioactive components they are only present in small quantities which would not be sufficient to provide the desired benefits to human brain function.

When skimmed milk powder is used as the predominant dairy component for a synthetic nutritional composition careful consideration of the type of protein being imparted to said composition is required. Skimmed milk powder provides predominantly casein protein, whereas nutritional compositions for human consumption, and particularly nutritional compositions intended for administration to paediatric subjects including children or infants, preferably possess a whey protein to casein protein ratio of 60:40. Therefore, to provide a synthetic nutritional composition that falls within the desired whey protein to casein protein ratio it is necessary to provide a source of whey protein, this is typically achieved using a whey protein concentrate.

Accordingly it is an object of the present invention to provide a synthetic nutritional composition for human consumption that possesses sufficient bioactive components to support cognitive development, particularly in infants and children, and to further ideally possess a predetermined whey protein to casein protein ratio.

### SUMMARY OF INVENTION

According to a first aspect of the present invention there is provided therefor a synthetic nutritional composition for human consumption comprising a whey protein concentrate from milk, wherein the whey protein concentrate comprises:
a milk fat content of between 6.5 to 10.0 wt %, wherein the milk fat contains components of milk fat globule membranes that provide at least 7 mg/g of sphingomyelin to the whey protein concentrate; and wherein the majority of the sphingomyelin in the synthetic nutritional composition is provided by the components of the milk fat globule membranes of the whey protein concentrate.

According to a second aspect of the present invention there is provided therefor a method of making a synthetic nutritional composition for human consumption comprising at least 0.7 mg/g of sphingomyelin on a dry weight basis; wherein the method comprises the blending of a whey protein concentrate with at least one other component of the nutritional composition or blending with a substantially complete nutritional composition or blending with an incomplete nutritional composition, wherein said blending is either dry blending or wet blending and wherein the whey protein concentrate comprises:
a milk fat content of between 6.5 to 10.0 wt %, wherein the milk fat contains components of milk fat globule membranes that provide at least 7 mg/g of sphingomyelin; and wherein the majority of the sphingomyelin in the synthetic nutritional composition is provided by the components of the milk fat globule membranes of the whey protein concentrate.

Milk fat globule membranes are a naturally occurring bioactive membrane structure that surrounds the fat droplets in the milk fat. Milk fat globule membranes are comprised of numerous components including, but not limited to, a trilayer lipid structure that comprises a complex mixture of phospholipids (such as phosphatidylethanolamine, phosphatidylcholine, phosphatidylserine, and phosphatidylinositol), glycolipids, glycosphingolipids (such as sphingomyelin and gangliosides), other polar lipids, proteins, glycoproteins (such as xanthine dehydrogenase, xanthine oxidase, lactadherin, fatty acid binding proteins (FABPs), mucin-1 (MUC-1), butyrophilins, adipophilin, and cluster of differentiation 36 (CD36)), triglycerides, cholesterol, 7-dehydrocholesterol, enzymes, and other components.

A significant body of preclinical data has demonstrated that milk fat globule membranes and/or components thereof, play roles in neural development and function, gastrointestinal immune defence and gut health. Glycosylated proteins (mucin-1, mucin-15, butyrophilin, and lactadherin) and glycosylated sphingolipids from milk fat globule membranes may promote the development of healthy infant gut microbiota by favouring beneficial Bifidobacterium species (Bourlieu et al., Current Opinion in Clinical Nutrition and Metabolic Care: March 2015, 18(2), 118-127).

Preferably the milk is bovine milk. The nutritional compositions of the present invention may be powdered compositions for dilution at point of use prior to oral administration to a human. Alternatively, the nutritional compositions of the present invention are provided in ready to use liquid form for oral administration to a human as required without the need to reconstitute and/or dilute. When the nutritional composition is provided in liquid form the units of the components of said composition are understood to refer to the nutritional composition on a dry weight basis.

The whey protein concentrate may be provided in any suitable form for making a synthetic nutritional composition, preferably the whey protein concentrate is provided in powdered form or provided in a liquid form. When the whey protein concentrate is provided in liquid form the units of the components of said liquid whey protein concentrate are understood to refer to the whey protein concentrate on a dry weight basis.

Commercially available whey protein concentrates contain a fat content of between about 1.0 to about 5.0 wt % of fat. It has long been understood within the field of creating synthetic nutritional compositions that it is highly desirable to ensure the whey protein concentrate does not have higher wt % of fat levels due to perceived negative impacts to the production of the whey protein concentrate and resultant properties of said whey protein concentrate.

During the cheese making process enzymes are added to liquid milk to produce curds and whey is a co-product of that process. The resultant liquid whey is removed from curd and is concentrated and purified into a whey protein concentrate using membrane processing, a form of ultrafiltration that uses porous membranes to eliminate bacteria and de-fat the liquid whey whilst allowing carbohydrates and minerals to be retained with the whey proteins. Retaining higher levels of fat in the whey protein concentrate is considered undesirable from a production perspective as higher levels of fat increase the likelihood of fouling the membranes used during the ultrafiltration processing. Higher levels of fat also tend to decrease overrun whipping as well as decreases foam stability. Higher levels of fat within the resultant whey protein concentrate tend to negatively impact flavour characteristics and, particularly importantly, reduce the shelf-life stability thereof. Accordingly, manufacturers of whey protein concentrate have a preference to recover the fat from the liquid whey and reintroduce the recovered fat into the next cheese production run where higher levels of fat are more desirous.

Hitherto the nutritional compositions of the present invention enhanced levels of sphingomyelin were provided to nutritional compositions through the inclusion of an enriched milk product in which the enrichment process had enhanced certain milk fat globule membrane (MFGM) components such as proteins, phospholipids and fats found in the MFGM. The enriched milk product possesses a fat content of between 14 to 20 wt %. The enriched milk product would be combined with one or more commercially available whey protein concentrates and non-fat milk powder, together with sources of carbohydrates, non-milk lipids, vitamins, minerals and the like to produce a nutritional composition.

Alternatively, prior to the nutritional compositions of the present invention, enhanced levels of sphingomyelin were provided to nutritional compositions through the inclusion of an enriched whey protein extract. The enriched whey protein extract comprises a whey protein concentrate that has been enriched with an alpha-lactalbumin which itself has been processed, typically, via acidification and subsequent protein precipitation separation to enhance levels of sphingomyelin. Although enriched whey protein extracts have enhanced levels of sphingomyelin they do not possess the broader complex mixture of components as found in milk fat globule membranes such as phospholipids, other polar lipids, proteins, glycoproteins, triglycerides, cholesterol, 7-dehydrocholesterol, enzymes and other components. The enriched whey protein extract could be combined with one or more commercially available whey protein concentrates and non-fat milk powder, together with sources of carbohydrates, non-milk lipids, vitamins, minerals and the like to produce a nutritional composition.

In contrast however, the nutritional compositions of the present invention and methods of making same provide an alternative route to provide enhanced levels enhanced levels of sphingomyelin as well as enhanced levels of milk fat globule membranes and the components thereof to a nutritional composition without the use of an enriched milk product and/or without an enriched whey protein extract.

Preferably an enriched milk product and/or an enriched whey protein extract are substantially excluded from the nutritional compositions of the present invention and methods of making same. Even more preferably an enriched milk product and/or an enriched whey protein extract are completely excluded from the nutritional compositions of the present invention and methods of making same.

Preferably the whey protein concentrate comprises a milk fat content of between 7.5 to 10.0 wt %. More preferably the whey protein concentrate comprises a milk fat content of between 8.5 to 10.0 wt %. Even more preferably the whey protein concentrate comprises a milk fat content of between 9.0 to 10.0 wt %. Most preferably the whey protein concentrate comprises a milk fat content of 9.5 wt % (+/−0.3 wt %).

Preferably the whey protein concentrate comprises a sphingomyelin content of between 8.0 to 10.7 mg/g. More preferably the whey protein concentrate comprises a sphingomyelin content of between 9.1 to 10.7 mg/g. Even more preferably the whey protein concentrate comprises a sphingomyelin content between 9.6 to 10.7 mg/g. Most preferably the whey protein concentrate comprises a sphingomyelin content of 10.2 mg/g (+/−0.3 mg/g).

The nutritional composition may comprise between 6.5 to 12.0 wt % of the whey protein concentrate on a dry weight basis, but preferably comprises between 7.9 to 10.3 wt % of the whey protein concentrate, and more preferably comprises between 7.9 to 9.3 wt % of the whey protein concentrate, and yet more preferably comprises between 7.9 to 8.8 wt % of the whey protein concentrate, and most preferably comprises 8.3 wt % (+/−0.3 wt %) of the whey protein concentrate.

For the avoidance of doubt the term “majority” is used within the context of the statements of the present invention to mean the majority relative to the total quantity of sphingomyelin in the synthetic nutritional composition. The components of the milk fat globule membranes of the whey protein concentrate may provide at least 65% of the sphingomyelin of the total amount of sphingomyelin in the nutritional composition. Preferably the components of the milk fat globule membranes of the whey protein concentrate provides at least 75% of the sphingomyelin of the total amount of sphingomyelin in the nutritional composition. More preferably the components of the milk fat globule membranes of the whey protein concentrate provides at least 85% of the sphingomyelin of the total amount of sphingomyelin in the nutritional composition. Most preferably the components of the milk fat globule membranes of the whey protein concentrate provides greater than 90% of the sphingomyelin of the total amount of sphingomyelin in the nutritional composition.

Preferably the nutritional composition comprises at least 0.74 mg/g of sphingomyelin on a dry weight basis; and more preferably comprises at least 0.78 mg/g of sphingomyelin; and most preferably provides at least 0.8 mg/g of sphingomyelin.

Preferably the nutritional composition comprises a whey protein concentrate containing components of the milk fat globule membranes that provide additional sphingolipids and/or phospholipids including at least one of phosphatidylcholine (PC), phosphatidylethanolamine (PE), phosphatidylserine (PS), phosphatidylinositol (PI), and/or derivatives thereof, and/or glycosphingolipids, and/or glycoproteins, and/or cholesterol. More preferably the whey protein concentrate contains components of the milk fat globule membranes that provide more than one additional phospholipid from the group of phosphatidylcholine (PC), phosphatidylethanolamine (PE), phosphatidylserine (PS), phosphatidylinositol (PI), and/or derivatives thereof; and/or one or more gangliosides.

The whey protein concentrate may comprise components of the milk fat globule membranes that provide a total phospholipids content of between 25 to 50 mg/g. Preferably the whey protein concentrate comprises components of the milk fat globule membranes that provide a total phospholipids content of between 30 to 45 mg/g; and more preferably comprises a total phospholipids content of between 35 to 40 mg/g; and most preferably comprises a total phospholipids content of 37.5 (+/−1.5) mg/g.

The whey protein concentrate may comprise components of the milk fat globule membranes that provide a certain gangliosides content, specifically the ganglioside GD3 in an amount of between 700 to 2000 mg/Kg. Preferably the whey protein concentrate comprises components of the milk fat globule membranes that provide the ganglioside GD3 in an amount of between 800 to 1900 mg/Kg; and more preferably comprises the ganglioside GD3 in an amount of between 900 to 1800 mg/Kg; and most preferably comprises the ganglioside GD3 in an amount of 1275 (+/−200) mg/Kg.

The whey protein concentrate may comprise milk fat globule membranes that provide a certain gangliosides content, specifically the ganglioside GM3 in an amount of between 10 to 50 mg/Kg. Preferably the whey protein concentrate comprises milk fat globule membranes that provide the ganglioside GM3 in an amount of between 15 to 40 mg/Kg; and more preferably comprises the ganglioside GM3 in an amount of between 19 to 32 mg/Kg; and most preferably comprises the ganglioside GM3 in an amount of 25 (+/−4) mg/Kg.

The milk fat globules of the milk fat globule membranes in the whey protein concentrate may have an average diameter of at least about 2 μm, and preferably an average diameter in the range of from about 2 μm to about 13 μm, and more preferably an average diameter in the range of from about 2.5 μm to about 10 μm, and even more preferably an average diameter in the range of from about 6 μm to about 6 μm. Without being bound by any particular theory, it is believed that milk fat globules of the aforementioned sizes are more accessible to lipases therefore leading to better lipid digestion.

The nutritional composition may have one or more prebiotics, preferably in the form of at least one human milk oligosaccharide (HMO) and/or polydextrose (PDX) and/or galactooligosaccharide (GOS). Prebiotics suitable for the nutritional compositions of the present invention include 2′-fucosyllactose (2FL), 3′-fucosyllactose (3FL), lacto-N-tetraose (LNT), lacto-N-neotetraose (LNnT), 6′-sialyllactose (6SL), and 3′-sialyllactose (3SL), and preferably in an amount on a dry weight basis of 1.5 to 4 g/Kg of 2FL, 0.1 to 1.5 g/Kg of 3FL, 0.1 to 4.2 g/Kg of LNT, 0.1 to 4.2 g/Kg of LNnT, 0.01 to 0.15 g/Kg of 3SL, and/or 0.05 to 0.45 g/Kg of 6SL.

Nutritional compositions according to the present invention may contain a total protein content in an amount of between 4 to 15 g/100 g on a dry weight basis, and preferably in an amount of between 5 to 15 g/100 g, and even more preferably in an amount of between 7 to 13 g/100 g.

The nutritional compositions according to the present invention preferably have a whey protein to casein protein ratio of between 60:40 to 100:0 on a dry weight basis; more preferably a whey protein to casein protein ratio of 60:40 to 80:20, and even more preferably a whey protein to casein protein ratio of 60:40 to 70:30; and most preferred a whey protein to casein protein ratio of 60:40.

The nutritional compositions of the present invention may comprise at least one additional protein source to the whey protein concentrate selected from at least one of: intact protein; partially hydrolysed protein; extensively hydrolysed protein; small peptides, amino acid(s); or any combination thereof. The additional protein source may be any used in the art, such as whole milk, non-fat milk, whey protein, casein, soy protein, hydrolysed protein, amino acids, and the like. Bovine milk protein sources may comprise, but are not limited to, whole milk powder, milk protein powders, milk protein concentrates, milk protein isolates, non-fat milk solids, non-fat milk, non-fat dry milk, casein, acid casein, caseinate (e.g. sodium caseinate, sodium calcium caseinate, calcium caseinate) or any combination thereof. Most preferably the at least one additional protein source comprises non-fat milk.

As noted above, the additional protein source of the nutritional compositions of the present invention may comprise partially hydrolysed protein, extensively hydrolysed protein, or a combination thereof. The hydrolysed proteins may be treated with enzymes to break down some or most of the proteins that cause adverse symptoms with the goal of reducing allergic reactions, intolerance, and sensitisation. These proteins may be hydrolysed by any method known in the art.

The nutritional composition of the present invention may comprise partially hydrolyzed protein, extensively hydrolyzed protein or a combination thereof, in which the whey protein concentrate and/or its liquid form is used as protein substrate or one of the protein substrates.

The nutritional composition may further comprise free amino acids as a protein equivalent source. The amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and any combination thereof. The amino acids may be branched chain amino acids. The amount of free amino acids in the nutritional composition may vary from about 1 to about 5 g/100 kcal.

The nutritional compositions according to the present invention preferably comprise a carbohydrate source. Any carbohydrate source conventionally found in nutritional compositions such as lactose, corn syrup, maltodextrin, starch and the like and mixtures thereof may be used, although the preferred source of a principal carbohydrate or carbohydrate component is lactose particularly for nutritional compositions for paediatric subjects including children or infants. Preferably the nutritional compositions according to the present invention contain a total carbohydrate content in an amount of between 20 to 75 g/100 g on a dry weight basis, and preferably in an amount of between 30 to 60 g/100 g.

A nutritional composition according to the present invention may contain a source of lipids beside the milk fat in the whey protein concentrate. The lipid source may be any lipid which is suitable for use in nutritional compositions and particularly nutritional compositions for paediatric subjects including children or infants. Preferably the nutritional compositions according to the present invention contain a total lipid content in an amount of between 10 to 30 g/100 g on a dry weight basis, and preferably in an amount of between 20 to 30 g/100 g.

A nutritional composition according to the present invention may contain a source of essential fatty acids, in particular a source of one or more long-chain polyunsaturated fatty acids would be preferred such as docosahexaenoic acid, arachidonic acid or a combination thereof.

The nutritional composition may also contain all vitamins and minerals understood to be essential in the daily diet and in nutritionally significant amounts. Minimum requirements have been established for certain vitamins and minerals. Examples of minerals, vitamins and other nutrients optionally present in the nutritional compositions may include vitamin A, vitamin B₁, vitamin B₂, vitamin B₆, vitamin B₁₂, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form. The presence and amounts of specific minerals and other vitamins will vary depending on the intended consumer whether it is an adult or a paediatric subject including a child or an infant.

Particularly where the nutritional composition is provided in ready to use liquid form, the nutritional composition may contain emulsifiers and stabilizers such as soy lecithin, citric acid esters of mono- and di-glycerides, and the like.

Preferably the nutritional composition further comprises at least one probiotic. More preferably the probiotic is selected from Bifidobacterium longum subsp. infantis, Bifidobacterium longum BB536 (BL999, ATCC: BAA-999), and Bifidobacterium animalis subsp. lactis BB-12 (DSM No. 10140) Lactobacillus rhamnosus GG (ATCC number 53103), or a combination thereof. The nutritional composition may include a probiotic in any suitable amount but preferably in an amount of from about 1×10⁴ cfu/100 kcal to about 1.5×10¹⁰ cfu/100 kcal. The probiotic(s) may be either non-viable or viable. The selected probiotic(s) may be cultured according to any suitable method and prepared for addition to the nutritional composition by freeze-drying or spray-drying for example. Alternatively, probiotic preparations can be bought from specialist suppliers in a suitable form for addition to nutritional compositions.

Preferably the nutritional composition further comprises lactoferrin and/or a source of beta glucan.

Preferably the method of making a synthetic nutritional composition for human consumption comprises the inclusion of between 6.5 to 12.0 wt % of the whey protein concentrate on a dry weight basis, and preferably comprises the inclusion of between 7.9 to 10.3 wt % of the whey protein concentrate, and more preferably comprises the inclusion of between 7.9 to 9.3 wt % of the whey protein concentrate, and even more preferably comprises the inclusion of between 7.9 to 8.8 wt % of the whey protein concentrate, and most preferably comprises the inclusion of 8.3 wt % (+/−0.3 wt %) of the whey protein concentrate.

Preferably the method of making a synthetic nutritional composition for human consumption comprises the inclusion of a whey protein concentrate having a milk fat content of between 7.5 to 10.0 wt % on a dry weight basis, and more preferably a milk fat content of between 8.5 to 10.0 wt %, and even more preferably a milk fat content of between 9.0 to 10.0 wt %, and most preferably a milk fat content of 9.5 wt % (+/−0.3 wt %).

Preferably the method of making a synthetic nutritional composition for human consumption provides the nutritional composition with at least 0.74 mg/g of sphingomyelin on a dry weight basis; and more preferably provides the nutritional composition with at least 0.78 mg/g of sphingomyelin; and most preferably provides the nutritional composition with at least 0.8 mg/g of sphingomyelin.

According to a further aspect of the present invention there is provided therefor a method of improving and/or supporting cognitive function and/or development in a human subject, comprising the step of administering to the subject a nutritional composition according to the first aspect of the present invention.

The improvement in cognitive function and/or development may be selected from an improvement in one or more of: executive function, memory, attention, mental fatigue and/or mental processing speed. The support in cognitive function and/or development may be selected from support of the normal functioning in one or more of: executive function, memory, attention, mental fatigue and/or mental processing speed.

### Definitions

“Milk” means a substance that has been drawn or extracted from the mammary gland of a mammal.

“Milk-based composition” means a composition comprising any milk-derived or milk-based product known in the art. For example, a “milk-based composition” may comprise bovine casein, bovine whey, bovine lactose, or any combination thereof.

“Enriched milk product” refers to a milk ingredient that has been enriched with MFGM and/or certain MFGM components to result in a product containing proteins and lipids found in the MFGM and possessing a fat content of between 14 to 20 wt %. By way of example, Lacprodan MFGM-10 available form Aria Foods Ingredients is a form of an enriched milk product.

“Enriched whey protein extract” refers to an enriched whey protein extract comprising a whey protein concentrate that has been enriched with an alpha-lactalbumin which itself has been processed, typically, via acidification and subsequent protein precipitation separation to enhance the levels of sphingomyelin. Examples of enriched whey protein extracts are described in US2020/0245669.

“Nutritional composition” means a substance or composition that satisfies at least a portion of a subject's nutrient requirements. “Nutritional composition(s)” may refer to liquids, powders, gels, pastes, solids, concentrates, suspensions, or ready-to-use forms of enteral formulas, oral formulas, formulas for infants, formulas for paediatric subjects, formulas for children, young child milks, and/or formulas for adults.

The term “synthetic” when applied to a composition, nutritional composition, or mixture means a composition, nutritional composition, or mixture obtained by biological and/or chemical means, which can be chemically identical to the mixture naturally occurring in mammalian milks. A composition, nutritional composition, or mixture is said to be “synthetic” if at least one of its components is obtained by biological (e.g. enzymatic) and/or chemical means.

“Adult” refers to a human that is 18 years of age or greater.

“Paediatric” means a human under 18 years of age. The term “paediatric subject” may refer to preterm infants, full-term infants, and/or children, as described below. A paediatric subject may be a human subject that is between birth and 8 years old. In another aspect, “paediatric subject” refers to a human subject between 1 and 6 years of age. Alternatively, “paediatric subject” refers to a human subject between 6 and 12 years of age.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes full-term infants, preterm infants, low birth weight infants, very low birth weight infants, and extremely low birth weight infants. “Preterm” means an infant born before the end of the 37^{th} week of gestation. “Full-term” means an infant born after the end of the 37^{th} week ofgestation.

“Child” means a subject ranging in age from 12 months to 13 years. A child may be a subject between the ages of 1 and 12 years old. In another aspect, the terms “children” or “child” may refer to subjects that are between one and about six years old. Alternatively, the terms “children” or “child” may refer to subjects that are between about seven and about 12 years old. The term “young child” means a subject ranging from 1 year to 3 years of age.

“Infant formula” means a composition that satisfies at least a portion of the nutrient requirements of an infant and is, ideally, capable of providing a sole source of nutrition to the infant.

“Follow-up formula” means a composition that satisfies at least a portion of the nutrient requirements of an infant from the 6^{th} month onwards, and for young children from 1 to 3 years of age.

“Young child milk”, in terms of the present disclosure, means a fortified milk-based beverage intended for children over one year of age (typically from one to six years of age). Young child milks are designed with the intent to serve as a complement to a diverse diet, to provide additional insurance that a child achieves continual, daily intake of all essential vitamins and minerals, macronutrients plus additional functional dietary components, such as non-essential nutrients that have purported health-promoting properties.

“Majority” is used herein in reference to the total amount of sphingomyelin in the nutritional composition and means more than 50% of the total amount of sphingomyelin in the nutritional composition.

“Amount” whether the amount is expressed as a wt/wt amount or expressed as a wt % amount or otherwise, it is to be understood that the amount is expressed on a dry weight basis unless it is clearly indicated otherwise.

The term “enteral” means deliverable through or within the gastrointestinal, or digestive, tract. “Enteral administration” includes oral feeding, intragastric feeding, transpyloric administration, or any other administration into the digestive tract. “Administration” is broader than “enteral administration” and includes parenteral administration or any other route of administration by which a substance is taken into a subject's body.

The term “substantially free” means containing less than a functional amount of the specified component, typically less than 0.1% by weight, and includes 0% by weight of the specified ingredient.

As applied to nutrients, the term “essential” refers to any nutrient that cannot be synthesised by the body in amounts sufficient for normal growth, so it must be supplied by the diet. The term “conditionally essential” as applied to nutrients means that the nutrient must be supplied by the diet when adequate amounts of the precursor compound is unavailable to the body for endogenous synthesis to occur.

The term “viable”, refers to live microorganisms. The term “non-viable” or “non-viable probiotic” refers to non-living probiotic microorganisms, their cellular components and/or metabolites thereof. Such a non-viable probiotic may have been heat-killed or otherwise inactivated but may still retain the ability to favourably influence the health of the host.

The amount of viable probiotic is detailed in CFUs, with the amount of non-viable probiotic disclosed as probiotic cell equivalents, wherein the term “probiotic cell equivalents” refers to the level of non-viable, non-replicating probiotics equivalent to an equal number of viable cells. The term “non-replicating” is to be understood as the amount of non-replicating microorganisms obtained from the same amount of replicating bacteria (CFU/g), including inactivated probiotics, fragments of DNA, cell wall, cytoplasmic compounds, etc. In other words, the quantity of non-living, non-replicating organisms is expressed in terms of CFU as if all the microorganisms were alive, regardless whether they are dead, non-replicating, inactivated, fragmented, etc. The probiotic source incorporated into the composition may comprise both viable CFUs and non-viable cell-equivalents.

The term “prebiotic” refers to a non-digestible food ingredient that beneficially affects the host by selectively stimulating the growth and/or activity of one or a limited number of bacteria in the digestive tract, which can improve the health of the host. Prebiotics exert health benefits, which may include, but are not limited to: selective stimulation of the growth and/or activity of one or a limited number of beneficial gut bacteria; stimulation of the growth and/or activity of ingested probiotic microorganisms; selective reduction in gut pathogens; and, favourable influence on gut short chain fatty acid profile. The prebiotic of the composition may be naturally-occurring, synthetic, or developed through the genetic manipulation of organisms and/or plants, whether such new source is now known or developed later.

The term “organism” refers to any contiguous living system, such as an animal, plant, fungus, or micro-organism.

“Lactoferrin” refers to lactoferrin that is produced by or obtained from a source other than human breast milk.

“Executive function” means the ability to recognise, evaluate, and make a choice among a variety of alternative options and strategies. The term encompasses goal-directed behaviour, planning and/or cognitive flexibility.

All percentages, parts, and ratios as used herein are detailed by weight of the total composition, unless otherwise specified. All amounts specified as administered “per day” may be delivered in a single unit dose, in a single serving, or in two or more doses or servings administered over the course of a 24 hour period.

All references to singular characteristics or limitations in the present disclosure shall include the corresponding plural characteristic or limitation, and vice versa, unless otherwise specified or clearly implied to the contrary, by the context in which the reference is made.

All combinations of method or process steps disclosed herein can be performed in any order, unless otherwise specified or clearly implied to the contrary, by the context in which the referenced combination is made.

The compositions and methods of the present disclosure can comprise, consist of, or consist essentially of any of the components described herein, as well as including any additional component useful in nutritional compositions.

### DETAILED DESCRIPTION

The present invention is directed toward the inventive realisation that it is possible to provide a synthetic nutritional composition for human consumption that possesses sufficient bioactive components including, but not limited to, sphingomyelin, to support cognitive development, particularly in infants and children, whilst ideally also possessing a predetermined whey protein to casein protein ratio. This inventive realisation can be achieved through the use of a novel whey protein concentrate in the formation of a synthetic nutrition composition wherein the whey protein concentrate comprises a milk fat content of between 6.5 to 10.0 wt %, wherein the milk fat contains components of milk fat globule membranes that provide at least 7 mg/g of sphingomyelin to the whey protein concentrate; and wherein the majority of the sphingomyelin in the synthetic nutritional composition is provided by the components of the milk fat globule membranes of the whey protein concentrate.

The whey protein concentrates contained in the nutritional compositions of the present invention may comprise a milk fat content of between 7.5 to 10.0 wt %, and preferably comprise a milk fat content of between 8.5 to 10.0 wt %, and more preferably comprise a milk fat content of between 9.0 to 10.0 wt %, and most preferably comprise a milk fat content of 9.5 wt % (+/−0.3 wt %).

The whey protein concentrates contained in the nutritional compositions of the present invention may comprise a sphingomyelin content of between 8.0 to 10.7 mg/g, and preferably comprises a sphingomyelin content of between 9.1 to 10.7 mg/g, and more preferably comprises a sphingomyelin content between 9.6 to 10.7 mg/g, and most preferably comprises a sphingomyelin content of 10.2 mg/g (+/−0.3 mg/g).

The nutritional compositions of the present invention may comprise between 6.5 to 12.0 wt % of the whey protein concentrate on a dry weight basis, but preferably comprises between 7.9 to 10.3 wt % of the whey protein concentrate, and more preferably comprises between 7.9 to 9.3 wt % of the whey protein concentrate, and yet more preferably comprises between 7.9 to 8.8 wt % of the whey protein concentrate, and most preferably comprises 8.3 wt % (+/−0.3 wt %) of the whey protein concentrate.

The components of the milk fat globule membranes of the whey protein concentrate may provide at least 65% of the sphingomyelin of the total amount of sphingomyelin in the nutritional composition. Preferably the components of the milk fat globule membranes of the whey protein concentrate provides at least 75% of the sphingomyelin of the total amount of sphingomyelin in the nutritional composition. More preferably the components of the milk fat globule membranes of the whey protein concentrate provides at least 85% of the sphingomyelin of the total amount of sphingomyelin in the nutritional composition. Most preferably the components of the milk fat globule membranes of the whey protein concentrate provides greater than 90% of the sphingomyelin of the total amount of sphingomyelin in the nutritional composition.

Preferably the nutritional composition comprises a whey protein concentrate that provides at least 0.74 mg/g of sphingomyelin to the nutritional composition on a dry weight basis; and more preferably provides at least 0.78 mg/g of sphingomyelin to the nutritional composition; and most preferably provides at least 0.8 mg/g of sphingomyelin to the nutritional composition.

The whey protein concentrate contained in the nutritional compositions of the present invention may further comprise components of the milk fat globule membranes that provide additional sphingolipids and/or phospholipids including at least one of phosphatidylcholine (PC), phosphatidylethanolamine (PE), phosphatidylserine (PS), phosphatidylinositol (PI), and/or derivatives thereof, and/or glycosphingolipids, and/or glycoproteins, and/or cholesterol. More preferably the whey protein concentrate contains components of the milk fat globule membranes that provide more than one additional phospholipid from the group of phosphatidylcholine (PC), phosphatidylethanolamine (PE), phosphatidylserine (PS), phosphatidylinositol (PI), and/or derivatives thereof; and/or one or more gangliosides.

The nutritional compositions of the present invention may have one or more prebiotics, preferably in the form of at least one HMO and/or polydextrose (PDX) and/or galactooligosaccharide (GOS). Prebiotics suitable for the nutritional compositions of the present invention include human milk oligosaccharides (HMOs), preferred HMOs include 2′-fucosyllactose (2FL), 3′-fucosyllactose (3FL), lacto-N-tetraose (LNT), lacto-N-neotetraose (LNnT), 6′-sialyllactose (6SL), and 3′-sialyllactose (3SL), and preferably in an amount of 1.5 to 4 g/Kg of 2FL, 0.1 to 1.5 g/Kg of 3FL, 0.1 to 4.2 g/Kg of LNT, 0.1 to 4.2 g/Kg of LNnT, 0.01 to 0.15 g/Kg of 3SL, 0.05 to 0.45 g/Kg of 6SL.

Nutritional compositions according to the present invention may contain a total protein content in an amount of between 4 to 15 g/100 g on a dry weight basis, and preferably in an amount of between 5 to 15 g/100 g, and even more preferably in an amount of between 7 to 13 g/100 g. The nutritional compositions may have a whey protein to casein protein ratio of between 60:40 to 100:0 on a dry weight basis; more preferably a whey protein to casein protein ratio of 60:40 to 80:20; more preferably a whey to casein ratio of 60:40 to 70:30; and most preferred a whey to casein ratio of 60:40.

The nutritional compositions of the present invention may comprise at least one additional protein source to the whey protein concentrate selected from at least one of: intact protein; partially hydrolysed protein; extensively hydrolysed protein; small peptides, amino acid(s); or any combination thereof. The additional protein source may be any used in the art, such as non-fat milk, whey protein, casein, soy protein, hydrolysed protein, amino acids, and the like. Bovine milk protein sources may comprise, but are not limited to, milk protein powders, milk protein concentrates, milk protein isolates, non-fat milk solids, non-fat milk, non-fat dry milk, casein, acid casein, caseinate (e.g. sodium caseinate, sodium calcium caseinate, calcium caseinate) or any combination thereof. Most preferably the at least one additional protein source comprises non-fat milk.

The additional protein source of the nutritional compositions of the present invention may comprise partially hydrolysed protein, extensively hydrolysed protein, or a combination thereof. The hydrolysed proteins may be treated with enzymes to break down some or most of the proteins that cause adverse symptoms with the goal of reducing allergic reactions, intolerance, and sensitisation. These proteins may be hydrolysed by any method known in the art.

The nutritional composition may further comprise free amino acids as a protein equivalent source. The amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and any combination thereof. The amino acids may be branched chain amino acids. The amount of free amino acids in the nutritional composition may vary from about 1 to about 5 g/100 kcal.

The nutritional compositions according to the present invention preferably comprise a carbohydrate source. Any carbohydrate source conventionally found in nutritional compositions such as lactose, glucose, fructose, maltodextrins, sucrose, starch, maltodextrin, maltose, fructooligosaccharides, corn syrup, high fructose corn syrup, dextrose, corn syrup solids, rice syrup solids, or any combination thereof, although the preferred source of the principal carbohydrate or carbohydrate component is lactose particularly for nutritional compositions for paediatric subjects including children or infants. Moreover the carbohydrate source may be provided as hydrolysed, partially hydrolysed and/or extensively hydrolysed carbohydrate, inclusion of same may be desirable due to their easy digestibility. More specifically, hydrolysed carbohydrates are less likely to contain allergenic epitopes. The nutritional compositions may comprise a carbohydrate source comprising hydrolysed or intact, naturally or chemically modified, starches sourced from corn, tapioca, rice, or potato, in waxy or non-waxy forms, such as hydrolysed corn starch.

Preferably the nutritional compositions according to the present invention contain a total carbohydrate content in an amount of between 20 to 75 g/100 g on a dry weight basis, and preferably in an amount of between 30 to 60 g/100 g.

A nutritional composition according to the present invention may contain a source of lipids beside the milk fat in the whey protein concentrate. The lipid source may be any lipid which is suitable for use in nutritional compositions and particularly nutritional compositions for paediatric subjects including children or infants. Suitable lipid sources for the nutritional composition may comprise animal sources, such as milk fat, butter, butter fat, or egg yolk lipid; marine sources, such as fish oils, marine oils, or single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, or wheat germ oil; medium chain triglyceride oils; emulsions and esters of fatty acids; or any combination thereof. Preferably the lipid source(s) used in the nutritional compositions of the present invention are vegetable and/or plant oils. Preferably the nutritional compositions according to the present invention contain a total lipid content in an amount of between 10 to 30 g/100 g on a dry weight basis, and preferably in an amount of between 20 to 30 g/100 g.

A nutritional composition according to the present invention may contain a source of essential fatty acids, in particular a source of one or more long-chain polyunsaturated fatty acids (LCPUFAs). Preferred LCPUFAs include docosahexaenoic acid (DHA), α-linoleic acid, γ-linoleic acid, linoleic acid, linolenic acid, eicosapentaenoic acid (EPA), arachidonic acid (ARA), or any combination thereof. Preferably, the composition comprises a source of LCPUFAs comprising DHA, ARA, or a combination thereof. The LCPUFAs are preferably provided in an amount of between 0.05 to 1.0 g/100 g

Alternatively or additionally the amount of LCPUFA in the nutritional compositions may be at least about 5 mg/100 kcal, and preferably in amount from 5 mg/100 kcal to 100 mg/100 kcal, and even more preferably in amount from 10 mg/100 kcal to 50 mg/100 kcal.

The nutritional compositions may comprise 5 mg/100 kcal to 80 mg/100 kcal of DHA, and preferably may comprise about 10 mg/100 kcal to about 20 mg/100 kcal of DHA, and more preferably may comprise about 15 mg/100 kcal to about 20 mg/100 kcal of DHA.

The nutritional compositions may comprise about 10 mg/100 kcal to about 100 mg/100 kcal of ARA, and preferably may comprise about 15 mg/100 kcal to about 70 mg/100 kcal of ARA, and more preferably may comprise about 20 mg/100 kcal to about 40 mg/100 kcal of ARA.

The nutritional compositions may comprise both DHA and ARA. The weight ratio of ARA:DHA may be between about 1:3 and about 9:1, and preferably the ratio of ARA:DHA may be from about 1:2 to about 4:1.

The nutritional compositions may comprise oils containing DHA and/or ARA. The source(s) of DHA and/or ARA may be any source known in the art such as marine oil, fish oil, single cell oil, egg yolk lipid, or brain lipid. The DHA and ARA may be sourced from single cell Martek oils, DHASCO® and ARASCO®, or variations thereof. The DHA and ARA may be in a natural form, provided that the remainder of the LCPUFA source does not result in any substantial deleterious effect on the infant. Alternatively or additionally, the DHA and ARA may be used in refined form.

The nutritional compositions may also contain all vitamins and minerals understood to be essential in the daily diet and in nutritionally significant amounts. Minimum requirements have been established for certain vitamins and minerals. Examples of minerals, vitamins and other nutrients optionally present in the nutritional compositions may include vitamin A, vitamin Bi, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form. The presence and amounts of specific minerals and other vitamins will vary depending on the intended consumer whether it is an adult or a paediatric subject including a child or an infant.

The nutritional compositions may comprise choline. Choline is a nutrient that is essential for normal function of cells. Choline is a precursor for membrane phospholipids and it accelerates the synthesis and release of acetylcholine, a neurotransmitter involved in memory storage. Without wishing to be bound by theory, it is believed that dietary choline and docosahexaenoic acid (DHA) act synergistically to promote the biosynthesis of phosphatidylcholine and thus, help promote synaptogenesis in human subjects. Additionally, choline and DHA act synergistically to promote dendritic spine formation, which is important in the maintenance of established synaptic connections. The nutritional composition may comprise choline in an amount of 80 to 200 mg/100 g.

The nutritional compositions may comprise inositol. The composition may comprise between about 10 mg/100 kcal and 40 mg/100 kcal. Alternatively, the composition may comprise between about 200 mg/L and about 300 mg/L of inositol.

The nutritional compositions may comprise one or more emulsifiers, as an emulsifier can increase the stability of the composition. The emulsifier may comprise, but is not limited to, egg lecithin, soy lecithin, alpha lactalbumin, monoglycerides, diglycerides, or any combination thereof. The nutritional compositions may comprise from about 0.5 wt % to about 1 wt % of emulsifier on a dry weight basis, and preferably comprise from about 0.7 wt % to about 1 wt % of emulsifier.

The nutritional compositions may comprise one or more preservatives, as a preservative can extend the shelf-life of the nutritional composition. The preservative may comprise, but is not limited to, potassium sorbate, sodium sorbate, potassium benzoate, sodium benzoate, calcium disodium EDTA, or any combination thereof. The nutritional compositions may comprise from about 0.1 wt % to about 1.0 wt % of a preservative on a dry weight basis based on the total dry weight of the nutritional composition. Preferably, the nutritional compositions may comprise from about 0.4 wt % to about 0.7 wt % of a preservative, based on the total dry weight of the nutritional composition.

The nutritional compositions may comprise one or more stabilisers, as a stabiliser can help preserve the structure of the composition. The stabiliser may comprise, but is not limited to, gum arabic, gum ghatti, gum karaya, gum tragacanth, agar, furcellaran, guar gum, gellan gum, locust bean gum, pectin, low methoxyl pectin, gelatine, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, or any combination thereof.

Preferably the nutritional composition further comprises a probiotic. More preferably, the probiotic comprises Bifidobacterium longum subsp. infantis, Bifidobacterium longum BB536 (BL999, ATCC: BAA-999), and Bifidobacterium animalis subsp. lactis BB-12 (DSM No. 10140) Lactobacillus rhamnosus GG (ATCC number 53103), or a combination thereof. The nutritional composition may include a probiotic in any suitable amount but preferably in an amount of from about 1×10⁴ cfu/100 kcal to about 1.5×10¹⁰ cfu/100 kcal. The probiotic(s) may be either non-viable or viable.

The selected probiotic(s) may be cultured according to any suitable method and prepared for addition to the nutritional composition by freeze-drying or spray-drying for example. Alternatively, probiotic preparations can be bought from specialist suppliers in a suitable form for addition to nutritional compositions.

The nutritional compositions may comprise lactoferrin. The lactoferrin may comprise human lactoferrin produced by a genetically modified organism, non-human lactoferrin, or a combination thereof. The non-human lactoferrin may comprise bovine lactoferrin (bLF), porcine lactoferrin, equine lactoferrin, buffalo lactoferrin, goat lactoferrin, murine lactoferrin or camel lactoferrin.

Lactoferrin may be present in the nutritional compositions in an amount of 0.01 to 2 g/100 g on a dry weight basis. The lactoferrin may be present in an amount of at least about 15 mg/100 kcal to about 300 mg/100 kcal.

The nutritional compositions may comprise a source of beta glucan. The source of β-glucan may comprise β-1,3-glucan. The amount of β-glucan present in the nutritional composition may be between 0.01 to 2 g/100 g on a dry weight basis. The β-glucan may be present in an amount of between 3 to 17 mg/100 kcal.

The nutritional composition of the present invention may support an improvement in cognitive function, said improvement being measureable in a clinical setting following consumption of the composition of the invention.

The improvement in cognitive function may be selected from an improvement in executive function, memory, attention, mental fatigue and/or mental processing speed.

Executive function is the ability to coordinate and integrate cognitive-perceptual processes in relation to time and space, determining how well a subject can recognise, evaluate and make a choice among a variety of alternative options and strategies. Skills that comprise executive function include attention, working memory, inhibitory control and cognitive flexibility.

The improvement in executive function may be an improvement measured by at least one standardised clinical neuropsychological test. Non-limiting examples of suitable neuropsychological tests are:
Wisconsin Card Sorting Test (WCST)—demonstrates mental flexibility
Stroop Task Test—demonstrates inhibitory control
Trail Making Test (TMT)—demonstrates mental flexibility
Clock Drawing Test (CDT)—demonstrates planning
Verbal Fluency Test (VFT) animals category—demonstrates verbal fluency
Verbal Fluency Test (VFT) F, A, S—demonstrates verbal fluency
Digits Forward and Backward subtests (WAIS-R or WAIS-III)—demonstrates working memory.

Preferably, the improvement in memory is selected from an improvement in working memory, spatial memory and/or episodic memory.

The improvement in attention may be demonstrated by measuring “Choice Reaction Time”, meaning that the subject is required to respond to one stimulus but to not respond to another. A non-limiting example of a suitable neuropsychological test is the Continuous Performance Test, in which the subject is asked to respond, as quickly as possible, to a rare stimulus that is embedded in a stream of ongoing similar stimuli. These tests assess the subject's ability to maintain attention and vigilance for the target stimulus and the ability to inhibit responses to the nontarget stimuli.

Mental fatigue is a condition triggered by prolonged cognitive activity. The improvement in mental fatigue may be demonstrated by the Cognitive Demand Battery.

The method of making a synthetic nutritional composition for human consumption comprising at least 0.7 mg/g of sphingomyelin on a dry weight basis; comprises the blending of a whey protein concentrate with at least one other component of the nutritional composition or blending with a substantially complete nutritional composition or blending with an incomplete nutritional composition, wherein said blending is either dry blending or wet blending and wherein the whey protein concentrate comprises:
a milk fat content of between 6.5 to 10.0 wt %, wherein the milk fat contains components of milk fat globule membranes that provide at least 7 mg/g of sphingomyelin; and
wherein the majority of the sphingomyelin in the synthetic nutritional composition is provided by the components of the milk fat globule membranes in the whey protein concentrate.

Preferably the method comprises addition of a sufficient quantity of the whey protein concentrate such that said whey protein concentrate comprises between 7.9 to 12.0 wt % of the finished nutritional composition on a dry weight basis. More preferably the whey protein concentrate is added in a sufficient quantity such that it comprises between 7.9 to 9.3 wt % of the finished nutritional composition on a dry weight basis, and yet more preferably comprises between 7.9 to 8.8 wt % of the finished nutritional composition, and most preferably comprises 8.3 wt % (+/−0.3 wt %) of the finished nutritional composition.

The method of creating the nutritional compositions of the present invention can be by any conventional method known in the art with the nutritional composition being provided in powdered form ready for dilution at point of use prior to oral administration to a human.

However, the nutritional composition may be provided in any form known in the art including a gel, a suspension, a paste, a solid, a liquid, a liquid concentrate, a reconstitutable milk substitute, or a ready-to-use liquid product. When the nutritional composition is provided in a powdered form, the powder may have a particle size in the range of 5 μm to 1500 μm, and preferably the particle size is in the range of 10 μm to 300 μm.

When the nutritional composition is provided in ready to use liquid form, the nutritional composition may contain emulsifiers and stabilizers such as soy lecithin, citric acid esters of mono- and di-glycerides, and the like.

The nutritional compositions of the present invention may be intended for a paediatric subject or an adult. The paediatric subject may be an infant or a child. The nutritional compositions may be a nutritional supplement, an adult's nutritional product, a children's nutritional product, an infant formula, a human milk fortifier, a toddler milk, or any other composition designed for an infant or a paediatric subject. The nutritional compositions may be provided in an orally-ingestible form, wherein the orally-ingestible comprises a food, a beverage, a tablet, a capsule, or a powder.

The nutritional compositions may be expelled directly into a human subject's intestinal tract. The nutritional compositions may be expelled directly into the gut. The nutritional compositions may be formulated to be consumed or administered enterally under the supervision of a physician.

The scope of the present invention is defined in the appended claims. It is to be understood that the skilled person may make amendments to the scope of the claims without departing from the scope of the present disclosure.

### EXAMPLE COMPOSITIONS

Example nutritional compositions are provided to illustrate some aspects of the synthetic nutritional compositions of the present invention but should not be interpreted as any limitation thereon. Other aspects within the scope of the claims herein will be apparent to one skilled in the art from the consideration of the specification or practice of the nutritional composition or methods disclosed herein. It is intended that the specification, together with the examples, be considered to be exemplary only, with the scope and spirit of the disclosure being indicated by the claims which follow the example. The skilled person in the art may vary the amount of the nutrients, and optional nutrients if present, within the prescribed ranges depending on the requirements of the resultant nutritional composition.

### Example 1—General Nutritional Composition (Powdered)

| | Total per 100 g | |
|---|---|---|
| Nutrient | Minimum | Maximum |
| Total Protein (g) | 4 | 15 |
| Fat (g) (not from WPC) | 10 | 30 |
| Carbohydrates (g) | 20 | 75 |
| WPC (g) with milk fat content of between | 6.5 | 12.0 |
| 6.5-10 wt % of WPC | | |
| [Sphingomyelin (mg)] | [70] | [128] |
| Optional further nutrients | | |
| LCPUFA (preferably DHA and/or ARA) (g) | 0.05 | 1.0 |
| GOS (g) | 0.1 | 5 |
| PDX (g) | 0.1 | 5 |
| Probiotic (CFU) | 1 × 10⁴ | 1.5 × 10¹⁰ |
| HMO (e.g. 2-FL) (g) | 0.005 | 1.0 |
| Linoleic acid (g) | 0.1 | 5 |
| Lactoferrin | 0.01 | 2 |
| Beta Glucan | 0.01 | 2 |
| Vitamin A (IU) | 100 | 2000 |
| Vitamin D (IU) | 50 | 500 |
| Vitamin E (IU) | 1 | 15 |
| Vitamin K (mcg) | 10 | 60 |
| Thiamin (mcg) | 50 | 500 |
| Riboflavin (mcg) | 100 | 900 |
| Vitamin B6 (mcg) | 50 | 500 |
| Vitamin B12 (mcg) | 0.5 | 2.0 |
| Niacin (mcg) | 1000 | 7500 |
| Folic acid (mcg) | 40 | 120 |
| Panthothenic acid (mcg) | 1000 | 3000 |
| Biotin (mcg) | 5 | 30 |
| Vitamin C (mg) | 25 | 100 |
| Choline (mg) | 80 | 200 |
| Calcium (mg) | 100 | 600 |
| Phosphorus (mg) | 80 | 400 |
| Magnesium (mg) | 20 | 80 |
| Sodium (mg) | 80 | 180 |
| Potassium (mg) | 200 | 800 |
| Chloride (mg) | 50 | 500 |
| Iodine (mcg) | 20 | 100 |
| Iron (mg) | 2 | 15 |
| Zinc (mg) | 1 | 10 |
| Manganese (mcg) | 50 | 120 |
| Copper (mcg) | 50 | 500 |

### Example 22—Nutritional Composition (Powdered) with WPC at ˜6.5 Wt % Milk Fat

| | Total per 100 g | |
|---|---|---|
| Nutrient | Minimum | Maximum |
| Total Protein (g) | 6.4 | 15 |
| Fat (g) (not from WPC) | 20 | 30 |
| Carbohydrates (g) | 30 | 60 |
| WPC (g) with milk fat content of ~6.5 wt % | 10.1 | 12.0 |
| of WPC | | |
| [Sphingomyelin (mg)] | [70] | [83.5] |
| Optional further nutrients | | |
| LCPUFA (preferably DHA and/or ARA) (g) | 0.05 | 1.0 |
| GOS (g) | 0.1 | 5 |
| PDX (g) | 0.1 | 5 |
| Probiotic (CFU) | 1 × 10⁴ | 1.5 × 10¹⁰ |
| HMO (e.g. 2-FL) (g) | 0.005 | 1.0 |
| Linoleic acid (g) | 0.1 | 5 |
| Lactoferrin | 0.01 | 2 |
| Beta Glucan | 0.01 | 2 |
| Vitamin A (IU) | 100 | 2000 |
| Vitamin D (IU) | 50 | 500 |
| Vitamin E (IU) | 1 | 15 |
| Vitamin K (mcg) | 10 | 60 |
| Thiamin (mcg) | 50 | 500 |
| Riboflavin (mcg) | 100 | 900 |
| Vitamin B6 (mcg) | 50 | 500 |
| Vitamin B12 (mcg) | 0.5 | 2.0 |
| Niacin (mcg) | 1000 | 7500 |
| Folic acid (mcg) | 40 | 120 |
| Panthothenic acid (mcg) | 1000 | 3000 |
| Biotin (mcg) | 5 | 30 |
| Vitamin C (mg) | 25 | 100 |
| Choline (mg) | 80 | 200 |
| Calcium (mg) | 100 | 600 |
| Phosphorus (mg) | 80 | 400 |
| Magnesium (mg) | 20 | 80 |
| Sodium (mg) | 80 | 180 |
| Potassium (mg) | 200 | 800 |
| Chloride (mg) | 50 | 500 |
| Iodine (mcg) | 20 | 100 |
| Iron (mg) | 2 | 15 |
| Zinc (mg) | 1 | 10 |
| Manganese (mcg) | 50 | 120 |
| Copper (mcg) | 50 | 500 |

### Example 3—Nutritional Composition (Powdered) with WPC at ˜10.0 Wt % Milk Fat

| | Total per 100 g | |
|---|---|---|
| Nutrient | Minimum | Maximum |
| Total Protein (g) | 4 | 15 |
| Fat (g) (not from WPC) | 20 | 30 |
| Carbohydrates (g) | 30 | 60 |
| WPC (g) with milk fat content of ~10.0 wt % | 6.5 | 12.0 |
| of WPC | | |
| [Sphingomyelin (mg)] | [70] | [128] |
| Optional further nutrients | | |
| LCPUFA (preferably DHA and/or ARA) (g) | 0.05 | 1.0 |
| GOS (g) | 0.1 | 5 |
| PDX (g) | 0.1 | 5 |
| Probiotic (CFU) | 1 × 10⁴ | 1.5 × 10¹⁰ |
| HMO (e.g. 2-FL) (g) | 0.005 | 1.0 |
| Linoleic acid (g) | 0.1 | 5 |
| Lactoferrin | 0.01 | 2 |
| Beta Glucan | 0.01 | 2 |
| Vitamin A (IU) | 100 | 2000 |
| Vitamin D (IU) | 50 | 500 |
| Vitamin E (IU) | 1 | 15 |
| Vitamin K (mcg) | 10 | 60 |
| Thiamin (mcg) | 50 | 500 |
| Riboflavin (mcg) | 100 | 900 |
| Vitamin B6 (mcg) | 50 | 500 |
| Vitamin B12 (mcg) | 0.5 | 2.0 |
| Niacin (mcg) | 1000 | 7500 |
| Folic acid (mcg) | 40 | 120 |
| Panthothenic acid (mcg) | 1000 | 3000 |
| Biotin (mcg) | 5 | 30 |
| Vitamin C (mg) | 25 | 100 |
| Choline (mg) | 80 | 200 |
| Calcium (mg) | 100 | 600 |
| Phosphorus (mg) | 80 | 400 |
| Magnesium (mg) | 20 | 80 |
| Sodium (mg) | 80 | 180 |
| Potassium (mg) | 200 | 800 |
| Chloride (mg) | 50 | 500 |
| Iodine (mcg) | 20 | 100 |
| Iron (mg) | 2 | 15 |
| Zinc (mg) | 1 | 10 |
| Manganese (mcg) | 50 | 120 |
| Copper (mcg) | 50 | 500 |

### Example 4—Nutritional Composition (Powdered) with WPC at ˜9.5 wt % Milk Fat (60:40

| | Total per 100 g | |
|---|---|---|
| Nutrient | Minimum | Maximum |
| Total Protein (g) | 4.4 | 15 |
| Fat (g) (not from WPC) | 20 | 30 |
| Carbohydrates (g) | 30 | 60 |
| WPC (g) with milk fat content of ~9.5 wt % | 6.9 | 12.0 |
| of WPC | | |
| [Sphingomyelin (mg)] | [70] | [122] |
| Optional further nutrients | | |
| LCPUFA (preferably DHA and/or ARA) (g) | 0.05 | 1.0 |
| GOS (g) | 0.1 | 5 |
| PDX (g) | 0.1 | 5 |
| Probiotic (CFU) | 1 × 10⁴ | 1.5 × 10¹⁰ |
| HMO (e.g. 2-FL) (g) | 0.005 | 1.0 |
| Linoleic acid (g) | 0.1 | 5 |
| Lactoferrin | 0.01 | 2 |
| Beta Glucan | 0.01 | 2 |
| Vitamin A (IU) | 100 | 2000 |
| Vitamin D (IU) | 50 | 500 |
| Vitamin E (IU) | 1 | 15 |
| Vitamin K (mcg) | 10 | 60 |
| Thiamin (mcg) | 50 | 500 |
| Riboflavin (mcg) | 100 | 900 |
| Vitamin B6 (mcg) | 50 | 500 |
| Vitamin B12 (mcg) | 0.5 | 2.0 |
| Niacin (mcg) | 1000 | 7500 |
| Folic acid (mcg) | 40 | 120 |
| Panthothenic acid (mcg) | 1000 | 3000 |
| Biotin (mcg) | 5 | 30 |
| Vitamin C (mg) | 25 | 100 |
| Choline (mg) | 80 | 200 |
| Calcium (mg) | 100 | 600 |
| Phosphorus (mg) | 80 | 400 |
| Magnesium (mg) | 20 | 80 |
| Sodium (mg) | 80 | 180 |
| Potassium (mg) | 200 | 800 |
| Chloride (mg) | 50 | 500 |
| Iodine (mcg) | 20 | 100 |
| Iron (mg) | 2 | 15 |
| Zinc (mg) | 1 | 10 |
| Manganese (mcg) | 50 | 120 |
| Copper (mcg) | 50 | 500 |

What is claimed is:

1. A synthetic nutritional composition for human consumption comprising a whey protein concentrate from milk, wherein the whey protein concentrate comprises:
a milk fat content of between 6.5 to 10.0 wt %, wherein the milk fat contains components of milk fat globule membranes that provide at least 7 mg/g of sphingomyelin to the whey protein concentrate; and
wherein the majority of the sphingomyelin in the synthetic nutritional composition is provided by the components of the milk fat globule membranes of the whey protein concentrate.

2. The synthetic nutritional composition according to claim 1, wherein the whey protein concentrate comprises a milk fat content of between 7.5 to 10.0 wt %.

3. The synthetic nutritional composition according to claim 1, wherein the whey protein concentrate comprises a milk fat content of between 8.5 to 10.0 wt %.

4. The synthetic nutritional composition according to claim 1, wherein the whey protein concentrate comprises a milk fat content of between 9.0 to 10.0 wt %.

5. (canceled)

6. The synthetic nutritional composition according to claim 1, wherein the whey protein concentrate comprises a sphingomyelin content of between 8.0 to 10.7 mg/g.

7. The synthetic nutritional composition according to claim 1, wherein the whey protein concentrate comprises a sphingomyelin content of between 9.1 to 10.7 mg/g.

8. The synthetic nutritional composition according to claim 1, wherein the whey protein concentrate comprises a sphingomyelin content between 9.6 to 10.7 mg/g.

9. (canceled)

10. The synthetic nutritional composition according to claim 1, wherein the nutritional composition comprises between 6.5 to 12.0 wt % of the whey protein concentrate on a dry weight basis.

11. The synthetic nutritional composition according to claim 1, wherein the nutritional composition comprises between 7.9 to 10.3 wt % of the whey protein concentrate on a dry weight basis.

12. The synthetic nutritional composition according to claim 1, wherein the nutritional composition comprises between 7.9 to 9.3 wt % of the whey protein concentrate on a dry weight basis.

13. The synthetic nutritional composition according to claim 1, wherein the nutritional composition comprises between 7.9 to 8.8 wt % of the whey protein concentrate on a dry weight basis.

14-17. (canceled)

18. The synthetic nutritional composition according to claim 1, wherein the components of the milk fat globule membranes of the whey protein concentrate provides greater than 90% of the sphingomyelin of the total amount of sphingomyelin in the nutritional composition.

19. The synthetic nutritional composition according to claim 1, wherein the nutritional composition comprises at least 0.74 mg/g of sphingomyelin on a dry weight basis.

20-21. (canceled)

22. The synthetic nutritional composition according to claim 1, wherein the whey protein concentrate comprises a total phospholipids content of between 25 to 50 mg/g.

23. (canceled)

24. The synthetic nutritional composition according to claim 1, wherein the whey protein concentrate comprises a ganglioside GD3 in an amount of between 700 to 2000 mg/Kg.

25-30. (canceled)

31. A method of making a synthetic nutritional composition for human consumption comprising at least 0.7 mg/g of sphingomyelin on a dry weight basis; wherein the method comprises:
blending a whey protein concentrate with at least one other component of the nutritional composition or blending with a substantially complete nutritional composition or blending with an incomplete nutritional composition, wherein said blending is either dry blending or wet blending and wherein the whey protein concentrate comprises:
a milk fat content of between 6.5 to 10.0 wt %, wherein the milk fat contains components of milk fat globule membranes that provide at least 7 mg/g of sphingomyelin to the whey protein concentrate; and
wherein the majority of the sphingomyelin in the synthetic nutritional composition is provided by the components of the milk fat globule membranes of the whey protein concentrate.

32. The method of making a synthetic nutritional composition according to claim 31, wherein the method comprises the addition of a sufficient quantity of the whey protein concentrate such that said whey protein concentrate comprises between 6.5 to 12.0 wt % of the finished nutritional composition on a dry weight basis.

33-35. (canceled)

36. The method of making a synthetic nutritional composition according to claim 31, wherein the whey protein concentrate has a milk fat content of between 7.5 to 10.0 wt %

37-39. (canceled)

40. The method of making a synthetic nutritional composition according to claim 31, wherein the nutritional composition is provided with at least 0.74 mg/g of sphingomyelin on a dry weight basis.

41-42. (canceled)

43. A method of supporting cognitive development in a human subject, comprising the step of:
administering to the subject a synthetic nutritional composition according to claim 1.

44. A method of improving cognitive development in a human subject, comprising the step of:
administering to the subject a synthetic nutritional composition according to claim 1.
